(19) United States
(12) Patent Application Publication
(10) Pub. No.: US 2026/0311101 A1
(43) Pub. Date: Oct. 8, 2026

(54) SYSTEM AND METHOD FOR DETERMINING AND CORRECTING HIGH COGNITIVE LOAD WHILE DRAFTING CODE

(71) Applicant: JPMorgan Chase Bank, N.A., New York, NY (US)

(72) Inventors: Abhinandan CHAUDHURY, Bengaluru (IN); Abdulsab ANKALAGI, Bangalore (IN); Kaushik SARKAR, Bengaluru (IN); Sudeepto Kumar ROY, Bengaluru (IN); Sachin ARORA, Bangalore (IN); Katie FITZGERALD, Manhattan, NY (US)

(73) Assignee: JPMorgan Chase Bank, N.A., New York, NY (US)

(21) Appl. No.: 19/213,734

(22) Filed: May 20, 2025

(30) Foreign Application Priority Data
Apr. 4, 2025 (IN) 202511033231

(51) Int. Cl. G06Q 10/0639; A61B 3/14; A61B 5/11; A61B 5/16

(52) U.S. Cl. CPC G06Q 10/06398; A61B 3/145; A61B 5/1124; A61B 5/16

(57) ABSTRACT

A methodology for determining and correcting high cognitive load while drafting code is provided. The method may include: receiving first biometric data of a code drafter while drafting a code on an interface, the first biometric data include at least typing speed based upon detected movement of keys on a keyboard of the interface while drafting the code; analyzing the first biometric data and the code for indicators of the code drafter experiencing excessive cognitive load relative to predetermined criteria while drafting the code; and generating at least one recommendation, in response to the analyzing, to improve the code being drafted by the code drafter. Corrective measures may include locking out the interface and/or modifying the code created during the period of excessive cognitive load.

### CROSS REFERENCE TO RELATED APPLICATIONS

This Application claims priority to Indian Patent Application No. 202511033231 entitled SYSTEM AND METHOD FOR DETERMINING AND CORRECTING HIGH COGNITIVE LOAD WHILE DRAFTING CODE FILED Apr. 4, 2025, the contents of which are expressly incorporated herein by reference in its entirety.

### TECHNICAL FIELD

This disclosure relates to methods and apparatuses for using an artificial intelligence/machine learning (AI/ML) model to determine a cognitive load index of code drafters while drafting code and to modify the code based on the same.

### BACKGROUND

The developments described in this section are known to the inventors. However, unless otherwise indicated, it should not be assumed that any of the developments described in this section qualify as prior art merely by virtue of their inclusion in this section, or that these developments are known to a person of ordinary skill in the art.

Traditional methods for coding involve a human code drafter assigned a project to draft code to perform a specified function.

A technical problem with traditional coding is that it is largely subjective to the skill and knowledge of the authoring code drafter. Assign the same project to two coder drafters of relatively common skill and knowledge, and the results may vary in the efficiency of the code from a perspective of computer resources and power requirements to execute the code, time to execute the code, and the amount of subsequent testing of the code due to errors in the inefficient code. For coders of different skill and knowledge, the gap can widen substantially.

Yet another subjective factor is the mental drain on the code drafter while coding, referred to herein as cognitive load. Code generated while the coder is tired or has been coding for an extended period is likely to be less efficient from a perspective of computer resources and power requirements to execute the code, time to execute the code, and testing of the code due to errors in the code. Simply stated, a rested code drafter can generate more efficient code than a code drafter who is under cognitive strain.

There is accordingly a need to detect when a code drafter is drafting code under high cognitive load, and to respond with corrective efforts to improve code efficiency by reducing the cognitive strain and/or modifying the code.

### SUMMARY

The present disclosure, through one or more of its various aspects, embodiments, and/or specific features or sub-components, provides, among other features, various systems, servers, devices, methods, media, programs, and platforms for using an AI/ML model to determine a cognitive load index of code drafters while drafting code and to modify the code based on the same.

According to an aspect of the disclosure, a method is provided. The method includes: receiving first biometric data of a code drafter while drafting a code on an interface, the first biometric data include at least typing speed based upon detected movement of keys on a keyboard of the interface while drafting the code; analyzing the first biometric data and the code for indicators of the code drafter experiencing excessive cognitive load relative to predetermined criteria while drafting the code; and generating at least one recommendation, in response to the analyzing, to improve the code being drafted by the code drafter.

The above aspect may have various optional features. The method may include: locking out the interface in response to: the at least one recommendation being that the code drafter needs to take a break from drafting code; and a predetermined period of time passed after generating the at least one recommendation during which the drafter continued to draft code on the interface. The code drafted by the code drafter may contain first code content programmed to provide at least one function, the method further including: identifying, based on the analyzing, at least a first portion of the first code content that was drafted while the drafter was experiencing excessive cognitive load; and transforming the first code content into second code content that provides the at least one function at lower reliance on computer resources and electrical power than the first code content; and executing the code with the second code content. The first biometric data may include receiving an image of the code drafter drafting the code, and the analyzing includes: converting the image into a Red-Green-Blue (RGB) image; and identifying, in the RGB image based on color contrast of eye features, an eye shape of the code drafter consistent with eye fatigue. The first biometric data may include hand movement as reflecting typing speed, typing rhythm, and/or typing patterns from typing on the interface, and the analyzing the first biometric data may include identifying frequent pauses, corrections, or hesitations in the typing. The analyzing the code may include analyzing frequency of errors made by the code drafter, types of errors made by the code drafter, and/or instances of backtracking. The analyzing the code may include analyzing lines of code, code churn, and a Halstead Complexity Measure. The method may include: receiving sleep biometric data from a night preceding a workday in which the code drafter while drafting code on an interface, and the analyzing is based in part on the sleep biometric data. The first biometric data may include hand movement as reflected by mouse, trackpad, and/or trackball movement while drafting the code.

According to another aspect of the disclosure, a system is provided is provided. The system includes a processor and a non-transitory computer readable memory storing instructions programmed to cooperate with a processor to cause the processor to perform operations. The operations include: receiving first biometric data of a code drafter while drafting a code on an interface, the first biometric data include at least typing speed based upon detected movement of keys on a keyboard of the interface while drafting the code; analyzing the first biometric data and the code for indicators of the code drafter experiencing excessive cognitive load relative to predetermined criteria while drafting the code; and generating at least one recommendation, in response to the analyzing, to improve the code being drafted by the code drafter.

The above aspect may have various optional features. The operations may include: locking out the interface in response to: the at least one recommendation being that the code drafter needs to take a break from drafting code; and a predetermined period of time passed after generating the at least one recommendation during which the drafter continued to draft code on the interface. The code drafted by the code drafter may contain first code content programmed to provide at least one function, the operations further including: identifying, based on the analyzing, at least a first portion of the first code content that was drafted while the drafter was experiencing excessive cognitive load; and transforming the first code content into second code content that provides the at least one function at lower reliance on computer resources and electrical power than the first code content; and executing the code with the second code content. The first biometric data may include receiving an image of the code drafter drafting the code, and the analyzing includes: converting the image into a Red-Green-Blue (RGB) image; and identifying, in the RGB image based on color contrast of eye features, an eye shape of the code drafter consistent with eye fatigue. The first biometric data may include hand movement as reflecting typing speed, typing rhythm, and/or typing patterns from typing on the interface, and the analyzing the first biometric data may include identifying frequent pauses, corrections, or hesitations in the typing. The analyzing the code may include analyzing frequency of errors made by the code drafter, types of errors made by the code drafter, and/or instances of backtracking. The analyzing the code may include analyzing lines of code, code churn, and a Halstead Complexity Measure. The operations may include: receiving sleep biometric data from a night preceding a workday in which the code drafter while drafting code on an interface, and the analyzing is based in part on the sleep biometric data. The first biometric data may include hand movement as reflected by mouse, trackpad, and/or trackball movement while drafting the code.

According to another aspect of the disclosure, a non-transitory computer readable media storing instructions programmed to cooperate with a processor to cause the processor to perform operations is provided. The operations include: receiving first biometric data of a code drafter while drafting a code on an interface, the first biometric data include at least typing speed based upon detected movement of keys on a keyboard of the interface while drafting the code; analyzing the first biometric data and the code for indicators of the code drafter experiencing excessive cognitive load relative to predetermined criteria while drafting the code; and generating at least one recommendation, in response to the analyzing, to improve the code being drafted by the code drafter.

The above aspect may have various optional features. The operations may include: locking out the interface in response to: the at least one recommendation being that the code drafter needs to take a break from drafting code; and a predetermined period of time passed after generating the at least one recommendation during which the drafter continued to draft code on the interface. The code drafted by the code drafter may contain first code content programmed to provide at least one function, the operations further including: identifying, based on the analyzing, at least a first portion of the first code content that was drafted while the drafter was experiencing excessive cognitive load; and transforming the first code content into second code content that provides the at least one function at lower reliance on computer resources and electrical power than the first code content; and executing the code with the second code content. The first biometric data may include receiving an image of the code drafter drafting the code, and the analyzing includes: converting the image into a Red-Green-Blue (RGB) image; and identifying, in the RGB image based on color contrast of eye features, an eye shape of the code drafter consistent with eye fatigue. The first biometric data may include hand movement as reflecting typing speed, typing rhythm, and/or typing patterns from typing on the interface, and the analyzing the first biometric data may include identifying frequent pauses, corrections, or hesitations in the typing. The analyzing the code may include analyzing frequency of errors made by the code drafter, types of errors made by the code drafter, and/or instances of backtracking. The analyzing the code may include analyzing lines of code, code churn, and a Halstead Complexity Measure. The operations may include: receiving sleep biometric data from a night preceding a workday in which the code drafter while drafting code on an interface, and the analyzing is based in part on the sleep biometric data. The first biometric data may include hand movement as reflected by mouse, trackpad, and/or trackball movement while drafting the code.

### BRIEF DESCRIPTION OF THE DRAWINGS

The present disclosure is further described in the detailed description which follows, in reference to the noted plurality of drawings, by way of non-limiting examples of preferred embodiments of the present disclosure, in which like characters represent like elements throughout the several views of the drawings.

FIG. 1 illustrates a computer system for implementing a method for using an AI/ML model in accordance with an embodiment.

FIG. 2 illustrates an exemplary diagram of a network environment with a device for using an AI/ML model in accordance with an embodiment.

FIG. 3 illustrates a system diagram for implementing a method for using an AI/ML model in accordance with an embodiment.

FIG. 4 illustrates an exemplary flow chart of a process for using an AI/ML model to determine a cognitive load index of code drafters while drafting code and to modify the code based on the same, in accordance with an embodiment.

FIG. 5 illustrates a model design that corresponds to an environment for using an AI/ML model to determine a cognitive load index of code drafters while drafting code and to modify the code based on the same, in accordance with an embodiment.

FIG. 6 illustrates a data flow that corresponds to a process for using an AI/ML model to determine a cognitive load index of code drafters while drafting code and to modify the code based on the same, in accordance with an embodiment.

### DETAILED DESCRIPTION

Through one or more of its various aspects, embodiments and/or specific features or sub-components of the present disclosure, are intended to bring out one or more of the advantages as specifically described above and noted below.

The examples may also be embodied as one or more non-transitory computer readable media having instructions stored thereon for one or more aspects of the present technology as described and illustrated by way of the examples herein. The instructions in some examples include executable code that, when executed by one or more processors, cause the processors to carry out steps necessary to implement the methods of the examples of this technology that are described and illustrated herein.

As is traditional in the field of the present disclosure, example embodiments are described, and illustrated in the drawings, in terms of functional blocks, units and/or modules. Those skilled in the art will appreciate that these blocks, units and/or modules are physically implemented by electronic (or optical) circuits such as logic circuits, discrete components, microprocessors, hard-wired circuits, memory elements, wiring connections, and the like, which may be formed using semiconductor-based fabrication techniques or other manufacturing technologies. In the case of the blocks, units and/or modules being implemented by microprocessors or similar, they may be programmed using software (e.g., microcode) to perform various functions discussed herein and may optionally be driven by firmware and/or software. Alternatively, each block, unit and/or module may be implemented by dedicated hardware, or as a combination of dedicated hardware to perform some functions and a processor (e.g., one or more programmed microprocessors and associated circuitry) to perform other functions. Also, each block, unit and/or module of the example embodiments may be physically separated into two or more interacting and discrete blocks, units and/or modules without departing from the scope of the inventive concepts. Further, the blocks, units and/or modules of the example embodiments may be physically combined into more complex blocks, units and/or modules without departing from the scope of the present disclosure.

References to any “example” herein (e.g., “for example”, “an example of”, by way of example” or the like) are to be considered non-limiting examples regardless of whether expressly stated or not.

Reference to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various features are described which may be features for some embodiments but not other embodiments.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, technical and scientific terms used herein have the meaning as commonly understood by one of ordinary skill in the art to which this disclosure pertains. In the case of conflict, the present document, including definitions will control.

A technical problem with traditional coding is that it is largely subjective to the skill and knowledge of the authoring code drafter. Assign the same project to two coder drafters of relatively common skill and knowledge, and the results may vary in the efficiency of the code from a perspective of computer resources and power requirements to execute the code, time to execute the code, and the amount of subsequent testing of the code due to errors in the inefficient code. For coders of different skill and knowledge, the gap can widen substantially.

Yet another subjective factor is the mental drain on the code drafter while coding, referred to herein as cognitive load. Code generated while the coder is tired or has been coding for an extended period is likely to be less efficient from a perspective of computer resources and power requirements to execute the code, time to execute the code, and testing of the code due to errors in the code. Simply stated, a rested code drafter can generate more efficient code than a code drafter who is under cognitive strain.

According to an aspect of the disclosure, a methodology is provided. The methodology includes: receiving first biometric data of a code drafter while drafting a code on an interface, the first biometric data include at least typing speed based upon detected movement of keys on a keyboard of the interface while drafting the code; analyzing the first biometric data and the code for indicators of the code drafter experiencing excessive cognitive load relative to predetermined criteria while drafting the code; and generating at least one recommendation, in response to the analyzing, to improve the code being drafted by the code drafter.

The above aspect may have various optional features. The methodology may include: locking out the interface in response to: the at least one recommendation being that the code drafter needs to take a break from drafting code; and a predetermined period of time passed after generating the at least one recommendation during which the drafter continued to draft code on the interface. The code drafted by the code drafter may contain first code content programmed to provide at least one function, the methodology further including: identifying, based on the analyzing, at least a first portion of the first code content that was drafted while the drafter was experiencing excessive cognitive load; and transforming the first code content into second code content that provides the at least one function at lower reliance on computer resources and electrical power than the first code content; and executing the code with the second code content. The first biometric data may include receiving an image of the code drafter drafting the code, and the analyzing includes: converting the image into a Red-Green-Blue (RGB) image; and identifying, in the RGB image based on color contrast of eye features, an eye shape of the code drafter consistent with eye fatigue. The first biometric data may include hand movement as reflecting typing speed, typing rhythm, and/or typing patterns from typing on the interface, and the analyzing the first biometric data may include identifying frequent pauses, corrections, or hesitations in the typing. The analyzing the code may include analyzing frequency of errors made by the code drafter, types of errors made by the code drafter, and/or instances of backtracking. The analyzing the code may include analyzing lines of code, code churn, and a Halstead Complexity Measure. The method may include: receiving sleep biometric data from a night preceding a workday in which the code drafter while drafting code on an interface, and the analyzing is based in part on the sleep biometric data. The first biometric data may include hand movement as reflected by mouse, trackpad, and/or trackball movement while drafting the code.

The above methodology provides a technical solution to the technical problem. The monitoring and recommendations to the code drafter while coding during periods of high cognitive load reduces errors and inefficiencies in the generated code from a perspective of computer resources and power requirements to execute the code, time to execute the code, and testing of the code due to errors in the code. Simply stated, a rested code drafter can generate more efficient code than a code drafter who is under cognitive strain. Locking out the interface provides a specific mechanism to prevent generation of inefficient or error prone code. Identifying and replacing code generated during periods of high cognitive load produces code that is less prone to error and more with lower demand for computer resources and power requirements to execute the code, less time to execute the code, and reduced testing of the code due to errors in the code. The generation of the RGB image to identify eye features consistent with fatigue provides a physiological way to identify high cognitive load.

Several definitions that apply throughout this disclosure will now be presented.
The terms “substantial”, “substantially” or the like are defined to be essentially conforming to the particular dimension, shape, or other feature that the term modifies, such that the component need not be exact. For example, “substantially cylindrical” means that the object resembles a cylinder, but can have one or more deviations from a true cylinder. The terms are used as a modifier to imply “approximate” rather than “perfect.” It is a term of approximation, not a term of degree.
The term “comprising” when utilized means “including, but not necessarily limited to”; it specifically indicates open-ended inclusion or membership in the so-described combination, group, series and the like.
The term “a” means “one or more” unless the context clearly indicates a single element.
The term “about” when used in connection with a numerical value means a variation consistent with the range of error in equipment used to measure the values, for which ±5% may be expected.
“First,” “second,” etc., re labels to distinguish components or blocks of otherwise similar names, but does not imply any sequence or numerical limitation.
“And/or” for two possibilities means either or both of the stated possibilities (“A and/or B” covers A alone, B alone, or both A and B take together), and when present with three or more stated possibilities means any individual possibility alone, all possibilities taken together, or some combination of possibilities that is less than all of the possibilities. The language in the format “at least one of A . . . and N” where A through N are possibilities means “and/or” for the stated possibilities (e.g., at least one A, at least one N, at least one A and at least one N, etc.).
When an element is referred to as being “connected,” or “coupled,” to another element, it can be directly connected or coupled to the other element or intervening elements may be present. By contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).
As used herein, the term “front”, “rear”, “left,” “right,” “top” and “bottom” or other terms of direction, orientation, and/or relative position are used for explanation and convenience to refer to certain features of this disclosure. However, these terms are not absolute, and should not be construed as limiting this disclosure.

Shapes as described herein are not considered absolute. As is known in the art, surfaces often have waves, protrusions, holes, recesses, etc. to provide rigidity, strength and functionality. All recitations of shape (e.g., cylindrical) herein are to be considered modified by “substantially” regardless of whether expressly stated in the disclosure or claims, and specifically accounts for variations in the art as noted above.

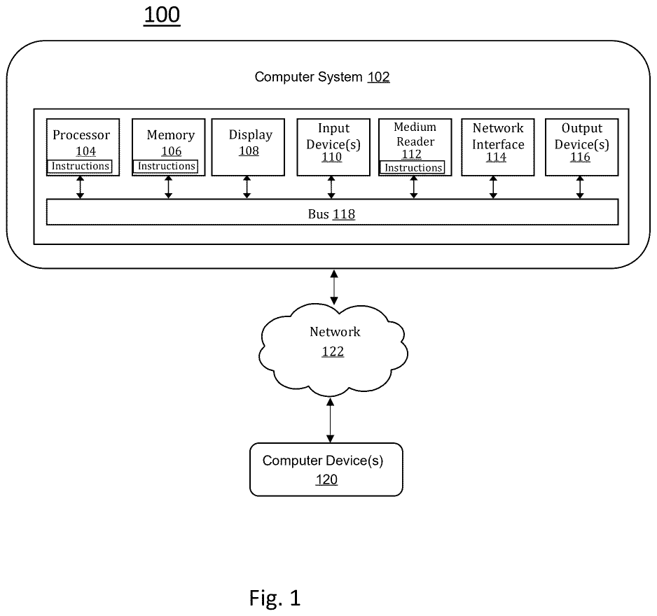

FIG. 1 is an exemplary system 100 for use in implementing a method for using an AI/ML model to determine a cognitive load index of code drafters while drafting code and to modify the code based on the same, in accordance with an embodiment. The system 100 is generally shown and may include a computer system 102, which is generally indicated.

The computer system 102 may include a set of instructions that may be executed to cause the computer system 102 to perform any one or more of the methods or computer-based functions disclosed herein, either alone or in combination with the other described devices. The computer system 102 may operate as a standalone device or may be connected to other systems or peripheral devices. For example, the computer system 102 may include, or be included within, any one or more computers, servers, systems, communication networks or cloud environment. Even further, the instructions may be operative in such cloud-based computing environment.

In a networked deployment, the computer system 102 may operate in the capacity of a server or as a client user computer in a server-client user network environment, a client user computer in a cloud computing environment, or as a peer computer system in a peer-to-peer (or distributed) network environment. The computer system 102, or portions thereof, may be implemented as, or incorporated into, various devices, such as a personal computer, a tablet computer, a set-top box, a personal digital assistant, a mobile device, a palmtop computer, a laptop computer, a desktop computer, a communications device, a wireless smart phone, a personal trusted device, a wearable device, a global positioning satellite (GPS) device, a web appliance, or any other machine capable of executing a set of instructions (sequential or otherwise) that specify actions to be taken by that machine. Further, while a single computer system 102 is illustrated, additional embodiments may include any collection of systems or sub-systems that individually or jointly execute instructions or perform functions. The term system shall be taken throughout the present disclosure to include any collection of systems or sub-systems that individually or jointly execute a set, or multiple sets, of instructions to perform one or more computer functions.

As illustrated in FIG. 1, the computer system 102 may include at least one processor 104. The processor 104 is tangible and non-transitory. As used herein, the term “non-transitory” is to be interpreted not as an eternal characteristic of a state, but as a characteristic of a state that will last for a period of time. The term “non-transitory” specifically disavows fleeting characteristics such as characteristics of a particular carrier wave or signal or other forms that exist only transitorily in any place at any time. The processor 104 is an article of manufacture and/or a machine component. The processor 104 is configured to execute software instructions in order to perform functions as described in the various embodiments herein. The processor 104 may be a general-purpose processor or may be part of an application specific integrated circuit (ASIC). The processor 104 may also be a microprocessor, a microcomputer, a processor chip, a controller, a microcontroller, a digital signal processor (DSP), a state machine, or a programmable logic device. The processor 104 may also be a logical circuit, including a programmable gate array (PGA) such as a field programmable gate array (FPGA), or another type of circuit that includes discrete gate and/or transistor logic. The processor 104 may be a central processing unit (CPU), a graphics processing unit (GPU), or both. Additionally, any processor described herein may include multiple processors, parallel processors, or both. Multiple processors may be included in, or coupled to, a single device or multiple devices.

The computer system 102 may also include a computer memory 106. The computer memory 106 may include a static memory, a dynamic memory, or both in communication. Memories described herein are tangible storage mediums that can store data and executable instructions, and are non-transitory during the time instructions are stored therein. Again, as used herein, the term “non-transitory” is to be interpreted not as an eternal characteristic of a state, but as a characteristic of a state that will last for a period of time. The term “non-transitory” specifically disavows fleeting characteristics such as characteristics of a particular carrier wave or signal or other forms that exist only transitorily in any place at any time. The memories are an article of manufacture and/or machine component. Memories described herein are computer-readable mediums from which data and executable instructions may be read by a computer. Memories as described herein may be random access memory (RAM), read only memory (ROM), flash memory, electrically programmable read only memory (EPROM), electrically erasable programmable read-only memory (EEPROM), registers, a hard disk, a cache, a removable disk, tape, compact disk read only memory (CD-ROM), digital versatile disk (DVD), floppy disk, or any other form of storage medium known in the art. Memories may be volatile or non-volatile, secure and/or encrypted, unsecure and/or unencrypted. Of course, the computer memory 106 may comprise any combination of memories or a single storage.

The computer system 102 may further include a display 108, such as a liquid crystal display (LCD), an organic light emitting diode (OLED), a flat panel display, a solid-state display, a cathode ray tube (CRT), a plasma display, or any other known display.

The computer system 102 may also include at least one input device 110, such as a keyboard, a touch-sensitive input screen or pad, a speech input, a mouse, a remote control device having a wireless keypad, a microphone coupled to a speech recognition module, a camera such as a video camera or still camera, a cursor control device, a GPS device, a visual positioning system (VPS) device, an altimeter, a gyroscope, an accelerometer, a proximity sensor, or any combination thereof. Those skilled in the art appreciate that various embodiments of the computer system 102 may include multiple input devices 110. Moreover, those skilled in the art further appreciate that the above-listed, exemplary input devices 110 are not meant to be exhaustive and that the computer system 102 may include any additional, or alternative, input devices 110.

The computer system 102 may also include a medium reader 112 which is configured to read any one or more sets of instructions, e.g., software, from any of the memories described herein. The instructions, when executed by a processor, may be used to perform one or more of the methods and processes as described herein. In a particular embodiment, the instructions may reside completely, or at least partially, within the memory 106, the medium reader 112, and/or the processor 104 during execution by the computer system 102.

Furthermore, the computer system 102 may include any additional devices, components, parts, peripherals, hardware, software, or any combination thereof which are commonly known and understood as being included with or within a computer system, such as, but not limited to, a network interface 114 and an output device 116. The output device 116 may be, but is not limited to, a speaker, an audio out, a video out, a remote control output, a printer, or any combination thereof.

Each of the components of the computer system 102 may be interconnected and communicate via a bus 118 or other communication link. As shown in FIG. 1, the components may each be interconnected and communicate via an internal bus. However, those skilled in the art appreciate that any of the components may also be connected via an expansion bus. Moreover, the bus 118 may enable communication via any standard or other specification commonly known and understood such as, but not limited to, peripheral component interconnect, peripheral component interconnect express, parallel advanced technology attachment, serial advanced technology attachment, etc.

The computer system 102 may be in communication with one or more additional computer devices 120 via a network 122. The network 122 may be, but is not limited to, a local area network, a wide area network, the Internet, a telephony network, a short-range network, or any other network commonly known and understood in the art. The short-range network may include, for example, infrared, near field communication, ultraband, or any combination thereof. Those skilled in the art appreciate that additional networks 122 which are known and understood may additionally or alternatively be used and that the exemplary networks 122 are not limiting or exhaustive. Also, while the network 122 is shown in FIG. 1 as a wireless network, those skilled in the art appreciate that the network 122 may also be a wired network.

The additional computer device 120 is shown in FIG. 1 as a personal computer. However, those skilled in the art appreciate that, in alternative embodiments of the present application, the computer device 120 may be a laptop computer, a tablet PC, a personal digital assistant, a mobile device, a palmtop computer, a desktop computer, a communications device, a wireless telephone, a personal trusted device, a web appliance, a server, or any other device that is capable of executing a set of instructions, sequential or otherwise, that specify actions to be taken by that device. Of course, those skilled in the art appreciate that the above-listed devices are merely exemplary devices and that the device 120 may be any additional device or apparatus commonly known and understood in the art without departing from the scope of the present application. For example, the computer device 120 may be the same or similar to the computer system 102. Furthermore, those skilled in the art similarly understand that the device may be any combination of devices and apparatuses.

Of course, those skilled in the art appreciate that the above-listed components of the computer system 102 are merely meant to be exemplary and are not intended to be exhaustive and/or inclusive. Furthermore, the examples of the components listed above are also meant to be exemplary and similarly are not meant to be exhaustive and/or inclusive.

In some embodiments, the modules implemented by the system 100 may be platform, language, database, and cloud agnostic that may allow for consistent easy orchestration and passing of data through various components to output a desired result regardless of platform, browser, language, database, and cloud environment by writing programs accordingly. The configuration or data files, in some embodiments, may be written using JavaScript Object Notation (JSON), but the disclosure is not limited thereto. For example, the configuration or data files may easily be extended to other readable file formats such as Extensible Markup Language (XML), YAML Ain't Markup Language (YAML), etc., or any other configuration-based languages.

In accordance with various embodiments of the present disclosure, the methods described herein may be implemented using a hardware computer system that executes software programs. Further, in a non-limited embodiment, implementations can include distributed processing, component/object distributed processing, and an operation mode having parallel processing capabilities. Virtual computer system processing may be constructed to implement one or more of the methods or functionality as described herein, and a processor described herein may be used to support a virtual processing environment.

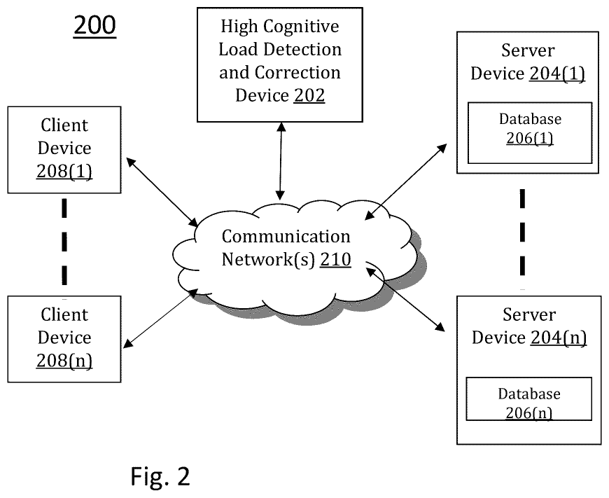

Referring to FIG. 2, a schematic of an exemplary network environment 200 for implementing a high cognitive load detection and correction device (HCLDACD) of the instant disclosure is illustrated.

In some embodiments, the above-described problems associated with conventional tools may be overcome by implementing an HCLDACD 202 as illustrated in FIG. 2 that may be configured for implementing a method for using an AI/ML model to determine a cognitive load index of code drafters while drafting code and to modify the code based on the same, but the disclosure is not limited thereto.

The HCLDACD 202 may have one or more computer system 102s, as described with respect to FIG. 1, which in aggregate provide the necessary functions.

The HCLDACD 202 may store one or more applications that can include executable instructions that, when executed by the HCLDACD 202, cause the HCLDACD 202 to perform actions, such as to transmit, receive, or otherwise process network messages, for example, and to perform other actions described and illustrated below with reference to the figures. The application(s) may be implemented as modules or components of other applications. Further, the application(s) may be implemented as operating system extensions, modules, plugins, or the like.

Even further, the application(s) may be operative in a cloud-based computing environment. The application(s) may be executed within or as virtual machine(s) or virtual server(s) that may be managed in a cloud-based computing environment. Also, the application(s), and even the HCLDACD 202 itself, may be located in virtual server(s) running in a cloud-based computing environment rather than being tied to one or more specific physical network computing devices. Also, the application(s) may be running in one or more virtual machines (VMs) executing on the HCLDACD 202. Additionally, in one or more embodiments of this technology, virtual machine(s) running on the HCLDACD 202 may be managed or supervised by a hypervisor.

In the network environment 200 of FIG. 2, the HCLDACD 202 is coupled to a plurality of server devices 204(1)-204(n) that hosts a plurality of databases 206(1)-206(n), and also to a plurality of client devices 208(1)-208(n) via communication network(s) 210. A communication interface of the HCLDACD 202, such as the network interface 114 of the computer system 102 of FIG. 1, operatively couples and communicates between the HCLDACD 202, the server devices 204(1)-204(n), and/or the client devices 208(1)-208(n), which are all coupled together by the communication network(s) 210, although other types and/or numbers of communication networks or systems with other types and/or numbers of connections and/or configurations to other devices and/or elements may also be used.

The communication network(s) 210 may be the same or similar to the network 122 as described with respect to FIG. 1, although the HCLDACD 202, the server devices 204(1)-204(n), and/or the client devices 208(1)-208(n) may be coupled together via other topologies. Additionally, the network environment 200 may include other network devices such as one or more routers and/or switches, for example, which are well known in the art and thus will not be described herein.

By way of example only, the communication network(s) 210 may include local area network(s) (LAN(s)) or wide area network(s) (WAN(s)), and can use TCP/IP over Ethernet and industry-standard protocols, although other types and/or numbers of protocols and/or communication networks may be used. The communication network(s) 210 in this example may employ any suitable interface mechanisms and network communication technologies including, for example, teletraffic in any suitable form (e.g., voice, modem, and the like), Public Switched Telephone Network (PSTNs), Ethernet-based Packet Data Networks (PDNs), combinations thereof, and the like.

The HCLDACD 202 may be a standalone device or integrated with one or more other devices or apparatuses, such as one or more of the server devices 204(1)-204(n), for example. In one particular example, the HCLDACD 202 may be hosted by one of the server devices 204(1)-204(n), and other arrangements are also possible. Moreover, one or more of the devices of the HCLDACD 202 may be in the same or a different communication network including one or more public, private, or cloud networks, for example.

The plurality of server devices 204(1)-204(n) may be the same or similar to the computer system 102 or the computer device 120 as described with respect to FIG. 1, including any features or combination of features described with respect thereto. For example, any of the server devices 204(1)-204(n) may include, among other features, one or more processors, a memory, and a communication interface, which are coupled together by a bus or other communication link, although other numbers and/or types of network devices may be used. The server devices 204(1)-204(n) in this example may process requests received from the HCLDACD 202 via the communication network(s) 210 according to the HyperText Transfer Protocol (HTTP)-based and/or JSON protocol, for example, although other protocols may also be used.

The server devices 204(1)-204(n) may be hardware or software or may represent a system with multiple servers in a pool, which may include internal or external networks. The server devices 204(1)-204(n) hosts the databases 206(1)-206(n) that are configured to store various types of data.

Although the server devices 204(1)-204(n) are illustrated as single devices, one or more actions of each of the server devices 204(1)-204(n) may be distributed across one or more distinct network computing devices that together comprise one or more of the server devices 204(1)-204(n). Moreover, the server devices 204(1)-204(n) are not limited to a particular configuration. Thus, the server devices 204(1)-204(n) may contain a plurality of network computing devices that operate using a master/slave approach, whereby one of the network computing devices of the server devices 204(1)-204(n) operates to manage and/or otherwise coordinate operations of the other network computing devices.

The server devices 204(1)-204(n) may operate as a plurality of network computing devices within a cluster architecture, a peer-to peer architecture, virtual machines, or within a cloud architecture, for example. Thus, the technology disclosed herein is not to be construed as being limited to a single environment and other configurations and architectures are also envisaged.

The plurality of client devices 208(1)-208(n) may also be the same or similar to the computer system 102 or the computer device 120 as described with respect to FIG. 1, including any features or combination of features described with respect thereto. Client device in this context refers to any computing device that interfaces to communications network(s) 210 to obtain resources from one or more server devices 204(1)-204(n) or other client devices 208(1)-208(n).

In some embodiments, the client devices 208(1)-208(n) in this example may include any type of computing device that can facilitate the implementation of the HCLDACD 202 that may efficiently provide a platform for implementing a method for using an AI/ML model to determine a cognitive load index of code drafters while drafting code and to modify the code based on the same, but the disclosure is not limited thereto.

The client devices 208(1)-208(n) may run interface applications, such as standard web browsers or standalone client applications, which may provide an interface to communicate with the HCLDACD 202 via the communication network(s) 210 in order to communicate user requests. The client devices 208(1)-208(n) may further include, among other features, a display device, such as a display screen or touchscreen, and/or an input device, such as a keyboard, for example.

Although the exemplary network environment 200 with the HCLDACD 202, the server devices 204(1)-204(n), the client devices 208(1)-208(n), and the communication network(s) 210 are described and illustrated herein, other types and/or numbers of systems, devices, components, and/or elements in other topologies may be used. It is to be understood that the systems of the examples described herein are for exemplary purposes, as many variations of the specific hardware and software used to implement the examples are possible, as may be appreciated by those skilled in the relevant art(s).

One or more of the devices depicted in the network environment 200, such as the HCLDACD 202, the server devices 204(1)-204(n), or the client devices 208(1)-208(n), for example, may be configured to operate as virtual instances on the same physical machine. For example, one or more of the HCLDACD 202, the server devices 204(1)-204(n), or the client devices 208(1)-208(n) may operate on the same physical device rather than as separate devices communicating through communication network(s) 210. Additionally, there may be more or fewer HCLDACDs 202, server devices 204(1)-204(n), or client devices 208(1)-208(n) than illustrated in FIG. 2. In some embodiments, the HCLDACD 202 may be configured to send code at run-time to remote server devices 204(1)-204(n), but the disclosure is not limited thereto.

In addition, two or more computing systems or devices may be substituted for any one of the systems or devices in any example. Accordingly, principles and advantages of distributed processing, such as redundancy and replication also may be implemented, as desired, to increase the robustness and performance of the devices and systems of the examples. The examples may also be implemented on computer system(s) that extend across any suitable network using any suitable interface mechanisms and traffic technologies, including by way of example only teletraffic in any suitable form (e.g., voice and modem), wireless traffic networks, cellular traffic networks, Packet Data Networks (PDNs), the Internet, intranets, and combinations thereof.

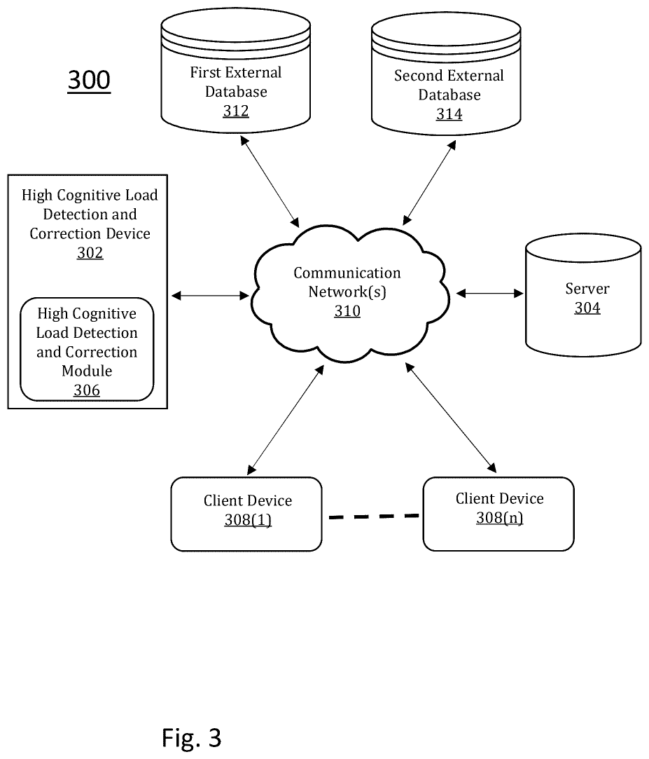

FIG. 3 illustrates a system diagram for implementing an HCLDACD 302 having a high cognitive load detection and correction module (HCLDACM), in accordance with an embodiment.

As illustrated in FIG. 3, the system 300 may include an HCLDACD 302 within which an HCLDACM 306 is embedded, a server 304, a first external database 312, a second external database 314, a plurality of client devices 308(1) . . . 308(n), and a communication network 310.

In some embodiments, the HCLDACD 302 including the HCLDACM 306 may be connected to the server 304, and the database(s) 312 via the communication network 310. The HCLDACD 302 may also be connected to the plurality of client devices 308(1) . . . 308(n) via the communication network 310, but the disclosure is not limited thereto.

In an embodiment, the HCLDACD 302 is described and shown in FIG. 3 as including the HCLDACM 306, although it may include other rules, policies, modules, databases, or applications, for example. In some embodiments, the first external database 312 and/or the second external database 314 may be configured to store ready to use modules written for each application programming interface (API) for all environments. Although only one database is illustrated in FIG. 3, the disclosure is not limited thereto. Any number of desired databases may be utilized for use in the disclosed invention herein. The databases 312, 314 may be a mainframe database, a log database that may produce programming for searching, monitoring, and analyzing machine-generated data via a web interface, etc., but the disclosure is not limited thereto.

In some embodiments, the HCLDACM 306 may be configured to receive real-time feed of data from the plurality of client devices 308(1) . . . 308(n) and secondary sources via the communication network 310.

The plurality of client devices 308(1) . . . 308(n) are illustrated as being in communication with the HCLDACD 302. In this regard, the plurality of client devices 308(1) . . . 308(n) may be “clients” (e.g., customers) of the HCLDACD 302 and are described herein as such. Nevertheless, it is to be known and understood that the plurality of client devices 308(1) . . . 308(n) need not necessarily be “clients” of the HCLDACD 302, or any entity described in association therewith herein. Any additional or alternative relationship may exist between either or both of the plurality of client devices 308(1) . . . 308(n) and the HCLDACD 302, or no relationship may exist.

The first client device 308(1) may be, for example, a smart phone. Of course, the first client device 308(1) may be any additional device described herein. The second client device 308(n) may be, for example, a personal computer (PC). Of course, the second client device 308(n) may also be any additional device described herein. In some embodiments, the server 304 may be the same or equivalent to the server device 204 as illustrated in FIG. 2.

The process may be executed via the communication network 310, which may comprise plural networks as described above. For example, in an embodiment, one or more of the plurality of client devices 308(1) . . . 308(n) may communicate with the HCLDACD 302 via broadband or cellular communication. Of course, these embodiments are merely exemplary and are not limiting or exhaustive.

The computing device 301 may be the same or similar to any one of the client devices 208(1)-208(n) as described with respect to FIG. 2, including any features or combination of features described with respect thereto. The HCLDACD 302 may be the same or similar to the HCLDACD 202 as described with respect to FIG. 2, including any features or combination of features described with respect thereto.

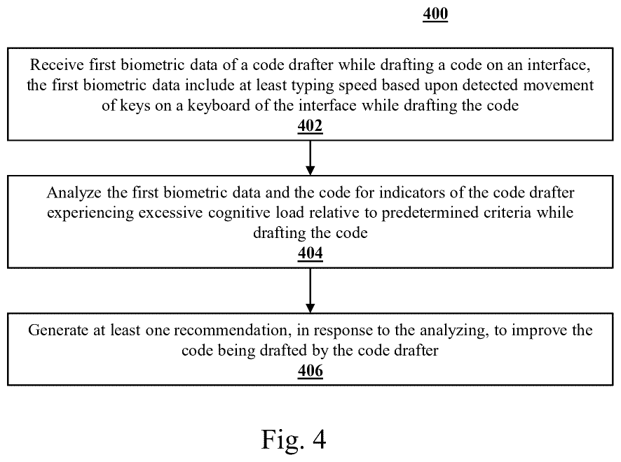

FIG. 4 illustrates an exemplary flow chart of a process 400 implemented by the HCLDACM 306 of FIG. 3 for enablement of a system and a method for using an AI/ML model to determine a cognitive load index of code drafters while drafting code and to modify the code based on the same, in accordance with an embodiment. It may be appreciated that the illustrated process 400 and associated steps may be performed in a different order, with illustrated steps omitted, with additional steps added, or with a combination of reordered, combined, omitted, or additional steps.

As illustrated in FIG. 4, at step S402, the process 400 may include receiving first biometric data of a code drafter while drafting a code on an interface. The first biometric data may include at least typing speed based upon detected movement of keys on a keyboard of the interface while drafting the code.

At step S404, the process 400 may include analyzing the first biometric data and the code for indicators of the code drafter experiencing excessive cognitive load relative to predetermined criteria while drafting the code; and

At step S406, the process 400 may include generating at least one recommendation, in response to the analyzing, to improve the code being drafted by the code drafter.

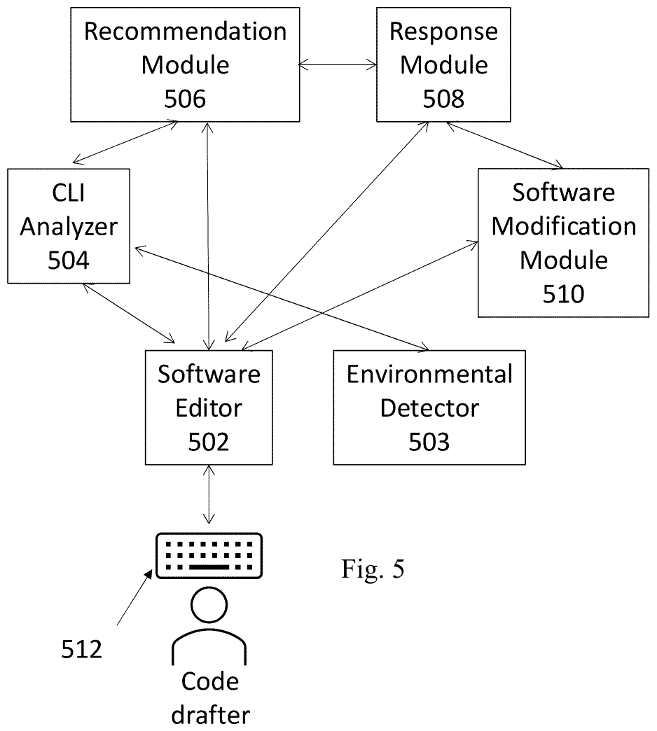

Referring now to FIG. 5, an environment 500 is shown. The environment includes a software code editor 502, at least one environmental detector 503, a Cognitive Load Index (CLI) analyzer 504, a recommendation module 506, a response module 508, and a software modification module 510.

The code drafter begins by interacting with the software code editor 502 on an appropriate computer interface 512 (e.g., computer, keyboard, mouse and the like) to draft code. This is where the code drafter writes, edits, and manages code. The software code editor 502 captures various user interactions, such as keystrokes, mouse movements, code changes, and navigation patterns.

The at least one environmental detector 503 monitors the environment of the code drafter. A non-limiting example of an environmental detector 503 is a camera that captures images of the code drafter's face and surrounding environment. Another non-limiting example is a smartwatch that monitors the code drafter's health status, such as sleeping patterns.

The software code editor 502 sends data about the code drafter's interactions to the CLI analyzer 504. The at least one environmental detector 503 also sends data about the code drafter's environment to the CLI analyzer 504. The CLI analyzer 504 processes the received data along with other criteria, such as the code itself, to assess the cognitive load experienced by the code drafter and generates a cognitive load score or profile which is then sent to recommendation module 506.

This recommendation module 506 uses the cognitive load score/profile, along with other contextual information, to generate personalized recommendations aimed at reducing cognitive load and improving coding efficiency.

Some recommendations generated by the recommendation module 506 may be sent back to the software code editor 502. These might include suggestions for code refactoring, reminders to take breaks, or tips on using specific editor features more effectively.

The software code editor 502 presents the received recommendations to the code drafter. This can be done through notifications, tooltips, or a dedicated recommendations panel within the software code editor 502.

The code drafter can then choose to implement these recommendations to enhance their coding workflow.

The response module 508 monitors the code drafter's response to the recommendations and take appropriate responsive action.

By way of non-limiting example, the recommendation may be that the code drafter needs to take a break from drafting code, yet the code drafter continues to draft code for a predetermined period of time (e.g., 10 minutes) after the recommendation. The response module 508 can respond by issuing a command to lock out the code drafter from the interface 512 for a predetermined period of time to either take a break (a few minutes break) or end their workday (a multi-hour break).

The recommendation module 506 may also determine that the code, or at least a portion of the code, drafted by the code drafter was drafted during a period while the drafter was experiencing excessive cognitive load as indicated by the cognitive load score/profile for that period. When code is drafted under an excess cognitive load, it is more likely that the code will contain errors that will increase downstream testing requirements, and/or that inefficiency of the code will cause the code to require excessive computer resources and power to operate and more time to execute. The recommendation module 506 can recommend that the code, or at least the noted portion of the code, be modified to reduce the likelihood of errors and/or to require less computer resources or electrical power to run.

The recommendation module may send a recommendation to modify the code to the software modification module 510. Software modification module 510 may be an AI/ML trained to revise code. By way of non-limiting example, if the code has a particular desired function, the software modification module 510 can redraft the code into a form that preserves the functionality but is less likely to have errors, and/or have an increased efficiency such that the new code requires less computer resources and electrical power than the original code of the drafter as generated during the period of excessive cognitive load.

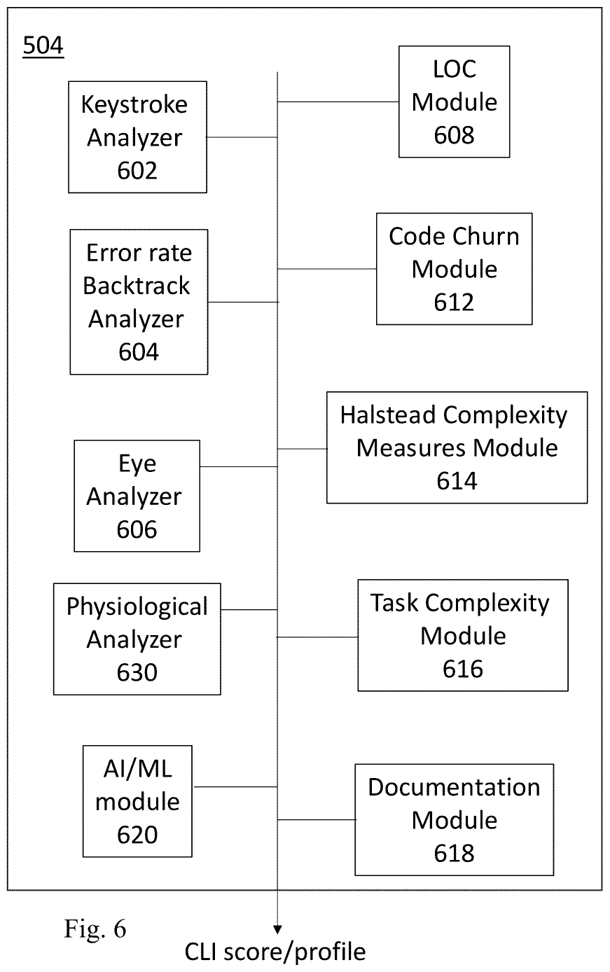

Referring now to FIG. 6, modules of the CLI analyzer 504 are shown in more detail. The modules may be categorized into code drafter biometrics modules and code specific modules. The CLI analyzer 504, which may be a trained AI/ML model or contain one, examines the collected data for indicators of excessive cognitive load relative to baseline standards for a group or individual code drafters.

Code drafter biometrics modules relate to analyzing specific movements, features, environment, and status of the code drafter. Code specific modules relate to analyzing specific features and characteristics of the code that the code drafter is drafting.

Non-limiting examples of code drafter biometric modules include a keystroke analyzer 602, an error rate and backtracking analyzer 604, an eye analyzer 606, and a physiological analyzer 630.

Keystroke analyzer 602 monitors and analyzes the code drafter's keystrokes while they are coding. It captures data such as typing speed, rhythm, and patterns. The keystroke analyzer 602 can provide insights into the code drafter's cognitive load and focus levels by identifying frequent pauses, corrections, or hesitations.

Keystroke analyzer 602 is driven by specific movements of when keys are pressed and released. When the code drafter pressed down on a key a “key down” event is triggered, and the exact time of this event is recorded. This timestamp represents the moment the key is initially pressed. When the code drafter releases the key that was previously pressed, the “key up” event is triggered, and the exact time of this event is recorded. This timestamp marks the end of the key press duration. Capturing the “key up” time allows for the calculation of the key press duration by comparing it with the “key down” time. This information allows for determining typing speed and rhythm. A logger/storage captures these events, which can store them in a structured format.

Keystrokes in this context may include pressing of keys on a standard computer keyboard as used to draft code. It may also include non-standard keyboards, number pads, and buttons/depressible areas on a mouse, trackball, and/or trackpad.

The system records both key up and key down event details in a storage logger in a structured format, which may include additional metadata such as the application context or user session information. The logger serves as a centralized repository for all keystroke data, enabling further analysis. Storing this data allows for historical tracking and can be used to identify patterns or trends in code drafter behavior.

Keystroke analyzer 602 can analyze a variety of factors to determine the cognitive load score/profile. One such example is the typing speed for the drafter relative to either an overall baseline speed or a previously observed baseline speed for the particular code drafter. If the code drafter is typing slower than normal, this is indicative that the code drafter is under excessive cognitive load.

Keystroke analyzer 602 can also monitor pressed keys for the number of times a particular key is pressed during a session or over a specified period, as well as how often a key is pressed within a given timeframe, providing insights into typing patterns and speed. It can also indicate which keys are used most frequently, which might suggest areas of focus or difficulty.

Error rate and backtracking analyzer 604 involves analyzing the frequency and types of errors made by the code drafter, as well as instances of backtracking (e.g., using the backspace key or undoing actions such as delete, cut, etc.). A high error rate or frequent backtracking can indicate areas of the code that are difficult to understand or implement, suggesting increased cognitive load or complexity.

Backtracking or similar key functions are monitored in the same manner as the keystroke analyzer 602 above, with specific focus on those keys or functions that relate to deleting and overwriting previously generated code. Frequent use of backspace might indicate areas where code drafters are making corrections, which could suggest difficulties with input or cognitive load.

Eye analyzer 606 involves isolating the eye in images of the code drafter and identifying indicia of fatigue or eye strain consistent with excessive cognitive load. A received image of the code drafted is converted into red-green-blue format (an RGB image). The conversion reduces noise in the original image and will allow for later color contrast within the image. A portion of the RGB image is created around one of the code drafter's eyes. The AI/ML model identifies within the portion physical characteristic consistent with fatigue or excessive cognitive load based on the shape or color(s) of the eye.

By way of non-limiting example, physical fatigue indicators include drooping eyelids, red or bloodshot eyes, and/or dark circles under the eye. All are eye conditions that can be determined from an RGB image of the eye area by examining the color contrast within the RGB image.

Physiologic analyzer 630 observes the code drafter's physiology for signs of cognitive load. Non-limiting examples may include sleep patterns from the night before, or current heart rate and blood pressure. Corresponding information may be provided by the environmental detector(s) 503.

CLI analyzer 504 may take into account typical physical behavior for the code drafter relative to the observed biometrics. By way of non-limiting example, code drafter A may type at 50 words per minute, while code drafter B may type at 30 words per minute. Observing a typing speed of 30 words per minute would be normal for code drafter B, but indicative of excessive cognitive load for code drafter A.

CLI analyzer 504 may also have code specific modules relate to analyzing specific features and characteristics of the code that the code drafter is drafting.

A Lines of Code (LOC) module 610 measures the total number of lines in the code. While not a direct measure of complexity, a higher LOC can indicate more work and potential complexity.

A Code Churn module 612 analyzes the amount of code that is added, modified, or deleted over time. High churn can indicate instability and complexity, as it suggests frequent changes.

A Halstead Complexity Measures module 614 analyzes the number of operators and operands in the code. They provide insights into the effort required to understand and maintain the code, including measures like volume, difficulty, and effort.

A Task Complexity module 616 synthesizes the data from the previous analyses to assess the overall complexity of the software development task. It considers factors such as the cognitive load indicated by keystrokes, error rates, code churn, and Halstead measures, etc. A high task complexity score might suggest the need for additional resources or time.

A Documentation module 618 determines, based on the assessed task complexity, the level and type of documentation needed. Complex tasks may require more detailed documentation to aid understanding and future maintenance. This can include inline comments, design documents, and user manuals.

The CLI analyzer 504, based on the above considerations and using a trained AI/ML model 620, generates a CLI score/profile that represents the cognitive load of the code drafter while drafting code during periods of time. The output could be in real-time for current status of the code drafter, a back looking report of past load, or a combination of both.

As discussed above, The CLI analyzer 504 generates a cognitive load score or profile which is then sent to recommendation module 506. The recommendation module 506 uses the cognitive load score/profile, along with other contextual information such as the underlying code, to generate specific recommendations aimed at reducing cognitive load and improving coding efficiency.

One type of recommendations is code quality and best practices:
Refactoring Suggestions: Recommend code refactoring opportunities to improve readability, maintainability, and performance.
Linting and Style Guide Compliance: Provide feedback on adherence to coding standards and style guides, suggesting corrections where necessary.
Error and Warning Alerts: Highlight potential errors, warnings, or anti-patterns in the code and suggest fixes.

Another type of recommendations is performance optimization:
Efficiency Improvements: Suggest optimizations for algorithms or data structures that could enhance performance.
Resource Usage: Provide insights into memory and CPU usage, recommending ways to reduce resource consumption.

Another type of recommendations is security enhancements:
Vulnerability Detection: Identify potential security vulnerabilities in the code and recommend mitigation strategies.
Dependency Updates: Alert developers to outdated or vulnerable dependencies and suggest updates.

Another type of recommendations is productivity and workflow:
Task Prioritization: Offer guidance on task prioritization based on deadlines, dependencies, or impact.
Time Management: Recommend time management techniques or tools to help developers stay organized and focused.

Another type of recommendations is collaboration and communication:
Code Review Participation: Encourage participation in code reviews, either as a reviewer or reviewee, to foster collaboration and knowledge sharing.
Documentation Suggestions: Recommend areas where additional documentation or comments could improve code understanding for team members.

Another type of recommendations is learning and skill development:
Learning Resources: Suggest tutorials, articles, or courses based on the developer's current work or areas of interest.
Skill Assessments: Provide opportunities for self-assessment or quizzes to help developers identify areas for improvement.

Another type of recommendations is well-being and work-life balance:
Break Reminders: Encourage regular breaks to prevent burnout and maintain focus.
Ergonomic Tips: Offer advice on ergonomic practices to reduce physical strain during long coding sessions.

Another type of recommendations is customization and personalization:
Tool and Plugin Recommendations: Suggest tools, plugins, or extensions that could enhance the developer's workflow or address specific needs.
Environment Configuration: Provide tips on configuring the development environment for optimal performance and comfort.

Another type of recommendations is feedback and continuous improvement:
Feedback Loops: Encourage developers to seek and provide feedback regularly to foster a culture of continuous improvement.
Retrospective Participation: Recommend participation in team retrospectives to reflect on past work and identify areas for growth.

Recommendations from the recommendation module 506 may be sent to the response module 508, which determines one or more appropriate responses to the recommendations.

One type of response from response module 508 is to send the code to software modification module 510, along with indicators of portions of the code generated while the code drafter was under high cognitive load. Software modification module 510 can analyze the identified portions of code for errors and replace it with code that lacks the errors, thereby reducing incidences of testing downstream when such errors may compound with other errors and require additional time and resources of full scale testing to identify. Software modification module 510 can analyze the identified portions of code for inefficiencies and replace the code with more efficient code, thereby providing code that reduces the strain on computer resources and electrical power and takes less time to run.

Another type of response from response module 508 is to send a notification to the code drafter's interface with the recommendation. The code drafter can then adopt or decline the recommendation, either directly (click an adopt/decline button), or indirectly (by following the recommendation or ignoring it).

Such adopt/decline may itself be further input to the CLI analyzer 504. For example, if the recommendation is for the code drafter to take a break and the code drafter continues coding, then CLI analyzer 504 may interpret this as further indicia of a high cognitive load, and response module 508 can take more aggressive steps such as lock out the interface 512 so the code drafter physically has to stop coding.

Another more aggressive step is for the response module to inform a supervisory authority of the high cognitive load. This can promote developer well-being and productivity by integrating a break notification system within the code interface 512. This system can encourage developers to take regular breaks and notify their managers if they continue working without taking a break. As discussed above, the methodology can continuously monitor code drafter activity within the interface 512, including keystrokes, mouse movements, and code changes.

The methodology can track the duration of continuous work sessions without breaks. After a predefined period of continuous work (e.g., 60 minutes), the interface 512 will display a gentle notification suggesting the developer take a short break. This notification can include tips on effective break activities, such as stretching, walking, or resting eyes.

If the developer continues to work for an additional period (e.g., 15 minutes) without taking a break, a secondary notification can be sent. This notification can inform the developer that their manager can be notified if they do not take a break soon.

If the developer still does not take a break after the secondary notification, an alert can be sent to the manager. The alert can include details such as the duration of continuous work and the developer's current workload. The manager can then decide on appropriate actions, such as checking in with the developer to ensure their well-being.

The methodology is customizable to management preferences. In one example, the cognitive load score/profile is compared related to customizable standards, which allows some environments to allow for more grinding than others. In another example, developers and managers can customize the break intervals and notification settings to suit individual preferences and team policies. Options can be provided to temporarily disable notifications during critical work periods, with a reminder to re-enable them afterward.

The methodology may have rules to ensure that all monitoring and notifications comply with data privacy regulations and ethical standards, and provide transparency to code drafters about what data is being collected and how it is used.

The methodology may utilize a variety of system components.

### Smart Devices and Sensors

Wearable Devices: Smartwatches, fitness bands, and/or smart rings can monitor physiological indicators such as heart rate, skin temperature, and galvanic skin response, which can be correlated with stress and cognitive load.
Environmental Sensors: Devices that measure ambient conditions like light, noise, and temperature can provide context about the developer's work environment, which can affect cognitive load.
Computer Vision: Cameras with computer vision capabilities can analyze facial expressions and posture to infer cognitive load and fatigue levels.

### Data Collection and Integration

APIs and Connectivity: Use APIs to collect data from smart devices and sensors. Ensure that the system can integrate data from multiple sources in real-time.
Data Aggregation: Aggregate data from various devices to create a comprehensive profile of the developer's cognitive load. This might include physiological data, environmental conditions, and behavioral patterns.

### Cognitive Load Index Analyzer:

Algorithm Development: Develop algorithms to process the aggregated data and calculate a Cognitive Load Index. This could involve machine learning models trained to recognize patterns associated with high cognitive load.
Thresholds and Alerts: Establish thresholds for different levels of cognitive load and configure the system to trigger alerts or notifications when these thresholds are exceeded.

### Feedback and Notification System

Real-time Feedback: Provide real-time feedback to developers through their smart devices or computer screens. This could include suggestions to take a break, adjust their environment, or perform relaxation exercises.
Personalized Recommendations: Use historical data to offer personalized recommendations for managing cognitive load, such as optimal break times or ergonomic adjustments.

### Privacy and Security

Data Privacy: Ensure that all data collection and processing comply with privacy regulations. Provide transparency to developers about what data is being collected and how it is used.
Secure Data Handling: Implement robust security measures to protect sensitive data from unauthorized access.

The above methodology provides a technical solution to the technical problem. The monitoring and recommendations to the code drafter while coding during periods of high cognitive load reduces errors and inefficiencies in the generated code from a perspective of computer resources and power requirements to execute the code, time to execute the code, and testing of the code due to errors in the code. Simply stated, a rested code drafter can generate more efficient code than a code drafter who is under cognitive strain. Locking out the interface provides a specific mechanism to prevent generation of inefficient or error prone code. Identifying and replacing code generated during periods of high cognitive load produces code that is less prone to error and more with lower demand for computer resources and power requirements to execute the code, less time to execute the code, and reduced testing of the code due to errors in the code. The generation of the RGB image to identify eye features consistent with fatigue provides a physiological way to identify high cognitive load.

In some embodiments as disclosed above in FIGS. 1-6, technical improvements effected by the instant disclosure may include a platform using an AI/ML model to determine a cognitive load index of code drafters while drafting code and to modify the code based on the same, but the disclosure is not limited thereto.

Although the invention has been described with reference to several exemplary embodiments, it is understood that the words that have been used are words of description and illustration, rather than words of limitation. Changes may be made within the purview of the appended claims, as presently stated and as amended, without departing from the scope and spirit of the present disclosure in its aspects. Although the invention has been described with reference to particular means, materials and embodiments, the invention is not intended to be limited to the particulars disclosed; rather the invention extends to all functionally equivalent structures, methods, and uses such as are within the scope of the appended claims.

For example, while the computer-readable medium may be described as a single medium, the term “computer-readable medium” includes a single medium or multiple media, such as a centralized or distributed database, and/or associated caches and servers that store one or more sets of instructions. The term “computer-readable medium” shall also include any medium that is capable of storing, encoding or carrying a set of instructions for execution by a processor or that cause a computer system to perform any one or more of the embodiments disclosed herein.

The computer-readable medium may comprise a non-transitory computer-readable medium or media and/or comprise a transitory computer-readable medium or media. In a particular non-limiting, exemplary embodiment, the computer-readable medium can include a solid-state memory such as a memory card or other package that houses one or more non-volatile read-only memories. Further, the computer-readable medium may be a random access memory or other volatile re-writable memory. Additionally, the computer-readable medium can include a magneto-optical or optical medium, such as a disk or tapes or other storage device to capture carrier wave signals such as a signal communicated over a transmission medium. Accordingly, the disclosure is considered to include any computer-readable medium or other equivalents and successor media, in which data or instructions may be stored.

Although the present application describes specific embodiments which may be implemented as computer programs or code segments in computer-readable media, it is to be understood that dedicated hardware implementations, such as application specific integrated circuits, programmable logic arrays and other hardware devices, may be constructed to implement one or more of the embodiments described herein. Applications that may include the various embodiments set forth herein may broadly include a variety of electronic and computer systems. Accordingly, the present application may encompass software, firmware, and hardware implementations, or combinations thereof. Nothing in the present application should be interpreted as being implemented or implementable solely with software and not hardware.

Although the present specification describes components and functions that may be implemented in particular embodiments with reference to particular standards and protocols, the disclosure is not limited to such standards and protocols. Such standards are periodically superseded by faster or more efficient equivalents having essentially the same functions. Accordingly, replacement standards and protocols having the same or similar functions are considered equivalents thereof.

The illustrations of the embodiments described herein are intended to provide a general understanding of the various embodiments. The illustrations are not intended to serve as a complete description of all of the elements and features of apparatus and systems that utilize the structures or methods described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, may be apparent to those of skill in the art upon reviewing the description.

The Abstract of the Disclosure is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. A method, comprising:
receiving first biometric data of a code drafter while drafting a code on an interface, the first biometric data include at least typing speed based upon detected movement of keys on a keyboard of the interface while drafting the code;
analyzing the first biometric data and the code for indicators of the code drafter experiencing excessive cognitive load relative to predetermined criteria while drafting the code; and
generating at least one recommendation, in response to the analyzing, to improve the code being drafted by the code drafter.

2. The method of claim 1, further comprising:
locking out the interface in response to:
the at least one recommendation being that the code drafter needs to take a break from drafting code; and
a predetermined period of time passed after generating the at least one recommendation during which the drafter continued to draft code on the interface.

3. The method of claim 1, wherein the code drafted by the code drafter contains first code content programmed to provide at least one function, the method further comprising:
identifying, based on the analyzing, at least a first portion of the first code content that was drafted while the drafter was experiencing excessive cognitive load;
transforming the first code content into second code content that provides the at least one function at lower reliance on computer resources and electrical power than the first code content; and
executing the code with the second code content.

4. The method of claim 1, wherein the first biometric data includes receiving an image of the code drafter drafting the code, and the analyzing comprises:
converting the image into a Red-Green-Blue (RGB) image; and
identifying, in the RGB image based on color contrast of eye features, an eye shape of the code drafter consistent with eye fatigue.

5. The method of claim 1, wherein the first biometric data includes hand movement as reflecting typing speed, typing rhythm, and/or typing patterns from typing on the interface, and the analyzing the first biometric data comprises identifying frequent pauses, corrections, or hesitations in the typing.

6. The method of claim 1, wherein the analyzing the code includes analyzing frequency of errors made by the code drafter, types of errors made by the code drafter, and/or instances of backtracking.

7. The method of claim 1, wherein the analyzing the code includes analyzing lines of code, code churn, and a Halstead Complexity Measure.

8. The method of claim 1, further comprising:
receiving sleep biometric data from a night preceding a workday in which the code drafter while drafting code on an interface; and
the analyzing is based in part on the sleep biometric data.

9. The method of claim 1, wherein the first biometric data includes hand movement as reflected by mouse, trackpad, and/or trackball movement while drafting the code.

10. A system, comprising:
a processor;
a non-transitory computer readable media storing instructions programmed to cooperate with a processor to cause the processor to perform operations, comprising:
receiving first biometric data of a code drafter while drafting a code on an interface, the first biometric data include at least typing speed based upon detected movement of keys on a keyboard of the interface while drafting the code;
analyzing the first biometric data and the code for indicators of the code drafter experiencing excessive cognitive load relative to predetermined criteria while drafting the code; and
generating at least one recommendation, in response to the analyzing, to improve the code being drafted by the code drafter.

11. The system of claim 10, the operations further comprising:
locking out the interface in response to:
the at least one recommendation being that the code drafter needs to take a break from drafting code; and
a predetermined period of time passed after generating the at least one recommendation during which the drafter continued to draft code on the interface.

12. The system of claim 10, wherein the code drafted by the code drafter contains first code content programmed to provide at least one function, the operations further comprising:
identifying, based on the analyzing, at least a first portion of the first code content that was drafted while the drafter was experiencing excessive cognitive load;
transforming the first code content into second code content that provides the at least one function at lower reliance on computer resources and electrical power than the first code content; and
executing the code with the second code content.

13. The system of claim 10, wherein the first biometric data includes receiving an image of the code drafter drafting the code, and the analyzing comprises:
converting the image into a Red-Green-Blue (RGB) image; and
identifying, in the RGB image based on color contrast of eye features, an eye shape of the code drafter consistent with eye fatigue.

14. The system of claim 10, wherein the first biometric data includes hand movement as reflecting typing speed, typing rhythm, and/or typing patterns from typing on the interface, and the analyzing the first biometric data comprises identifying frequent pauses, corrections, or hesitations in the typing.

15. The system of claim 10, wherein the analyzing the code includes analyzing frequency of errors made by the code drafter, types of errors made by the code drafter, and/or instances of backtracking.

16. The system of claim 10, wherein the analyzing the code includes analyzing lines of code, code churn, and a Halstead Complexity Measure.

17. The system of claim 10, the operations further comprising:
receiving sleep biometric data from a night preceding a workday in which the code drafter while drafting code on an interface; and
the analyzing is based in part on the sleep biometric data.

18. The system of claim 10, wherein the first biometric data includes hand movement as reflected by mouse, trackpad, and/or trackball movement while drafting the code.

19. A non-transitory computer readable media storing instructions programmed to cooperate with a processor to cause the processor to perform operations, comprising:
receiving first biometric data of a code drafter while drafting a code on an interface, the first biometric data include at least typing speed based upon detected movement of keys on a keyboard of the interface while drafting the code;
analyzing the first biometric data and the code for indicators of the code drafter experiencing excessive cognitive load relative to predetermined criteria while drafting the code; and
generating at least one recommendation, in response to the analyzing, to improve the code being drafted by the code drafter.

20. The method of claim 1, the operations further comprising:
locking out the interface in response to:
the at least one recommendation being that the code drafter needs to take a break from drafting code; and
a predetermined period of time passed after generating the at least one recommendation during which the drafter continued to draft code on the interface.
